(19) United States
(12) Patent Application Publication
(10) Pub. No.: US 2012/0187001 A1
(43) Pub. Date: Jul. 26, 2012

(54) HAND-HELD TEST METER WITH DEEP POWER CONSERVATION MODE VIA DIRECT OR GENERATED SIGNAL APPLICATION AND METHOD FOR EMPLOYING SUCH A METER

(75) Inventors: Ulrich KRAFT, Hofheim (DE); Rossano Massari, Milan (IT)

(73) Assignee: LifeScan, Inc., Milpitas, CA (US)

(21) Appl. No.: 13/014,453

(22) Filed: Jan. 26, 2011

(51) Int. Cl. G01N 33/66; G06F 13/00; G01N 27/327; G05B 19/00

(52) U.S. Cl. 205/792; 700/297; 710/305; 204/403.02

(57) ABSTRACT

A hand-held test meter for use with an analytical test strip in the determination of an analyte (such as glucose) in a bodily fluid sample (for example, a whole blood sample) includes a housing, a buttons electrical circuit block, at least one user operable button in operable communication with the buttons electrical circuit block, a microcontroller block, and a first-time-on (FTO) electrical circuit block. The FTO electrical circuit block is disposed within the housing and includes an activation node and a signal reception contact. In addition, the FTO electrical circuit block is configured to place the hand-held test meter into a deep power conservation mode upon either the direct application of an electrical signal to the activation node by an external device (e.g., a manufacturing tester) or a deactivation signal received at the signal reception contact. The FTO electrical circuit block is also configured to terminate the deep power conservation mode and place the hand-held test meter into a normal operating mode upon receiving a predetermined user triggered signal from the at least one user operable button. Moreover, the microcontroller block is configured to generate the deactivation signal received at the signal reception contact in response to an external command signal received by the microcontroller block.

### BACKGROUND OF THE INVENTION

1. Field of the Invention

The present invention relates, in general, to medical devices and, in particular, to test meters and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using a hand-held test meter in combination with analytical test strips (e.g., electrochemical-based analytical test strips).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified top view of a hand-held test meter according to an embodiment of the present invention;

FIG. 2 is a simplified block diagram of various blocks of the hand-held test meter of FIG. 1;

FIG. 3 is a simplified combined electrical schematic and block diagram of a first-time-on (FTO) electrical circuit block (within the dashed lines of FIG. 3), a buttons electrical circuit block, a power supply circuitry block, a microcontroller block and a battery as can be employed in embodiments of the present invention; and

FIG. 4 is a flow diagram depicting stages in a method for employing a hand-held test meter according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, hand-held test meters for use with an analytical test strip (e.g., an electrochemical-based analytical test strip) in the determination of an analyte (such as glucose) in a bodily fluid sample (for example, a whole blood sample) according to embodiments of the present invention include a housing, a buttons electrical circuit block, at least one user operable button in operable communication with the buttons electrical circuit block, a microcontroller block and a first-time-on (FTO) electrical circuit block.

In such hand-held test meters, the FTO electrical circuit block is disposed within the housing and includes an activation node and a signal reception contact. In addition, the FTO electrical circuit block is configured to place the hand-held test meter into a deep power conservation mode upon either (i) the direct application of an electrical signal to the activation node by an external device (e.g., a manufacturing tester) or (ii) a deactivation signal received at the signal reception contact. The FTO electrical circuit block is also configured to terminate the deep power conservation mode and place the hand-held test meter into a normal operating mode upon receiving a predetermined user triggered signal from the at least one user operable button. Moreover, the microcontroller block is configured to generate the deactivation signal received at the signal reception contact in response to an external command signal (for example, an automated test equipment (ATE) generated software command signal) received by the microcontroller block.

Hand-held test meters according to embodiments of the present invention are particularly beneficial in that they possess the flexibility and convenience of being configured for placement into the deep power conservation mode via either of two techniques, namely (i) the direct application an electrical signal to the FTO electrical circuit block or (ii) the receipt of a generated deactivation signal by the FTO electrical circuit block. For example, if the former of these techniques is inconvenient due to lack of ready access to the activation node during manufacturing, production testing or elsewhere in a supply chain, the latter technique can be employed. For example, the generated deactivation signal can be employed to conveniently and readily place a hand-held test meter into the deep power conservation mode following an update to firmware of the hand-held test meter. Due to the provision of these two techniques, one based on a directly applied signal and one based on a generated signal, hand-held test meters according to embodiments of the present invention are also referred to as hand-held test meters with deep power conservation mode via either direct or generated signal application.

Moreover, hand-held test meters according to embodiments of the present invention are also beneficial in that the deep power conservation mode cannot be inadvertently activated by an end user (i.e., a health care professional demonstrating the hand-held test meter or a patient operating the hand-held test meter) since both the activation node (also referred to as a test point) and the signal reception contact (which can take any suitable form including an electrical trace/wire) are disposed within the housing and not reasonably accessible to an end-user. However, since the predetermined user triggered signal can be generated by an end-user's normal operation of the hand-held test meter including, for example, simply turning on (activating) the hand-held test meter by pushing an appropriate hand-held test meter button, termination of the deep power conservation mode is simple, intuitive and requires no dedicated actions on the part of an end user. In addition, the deep power conservation mode enables shipment and prolonged storage of the hand-held test meter with a sealed rechargeable battery in a charged state without deleterious loss of charge. The hand-held meter is, therefore, ready for immediate operation (for example, an out-of-the-box test and demonstration) once the deep power conservation mode is terminated.

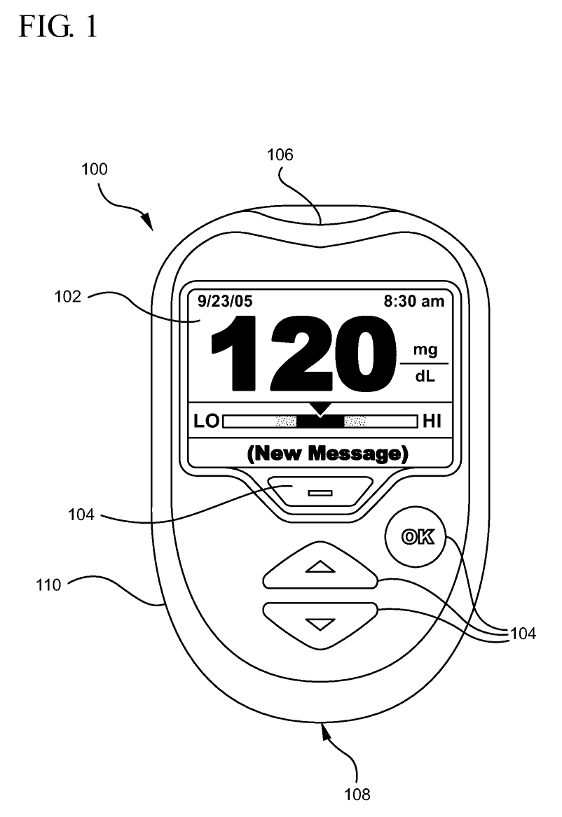

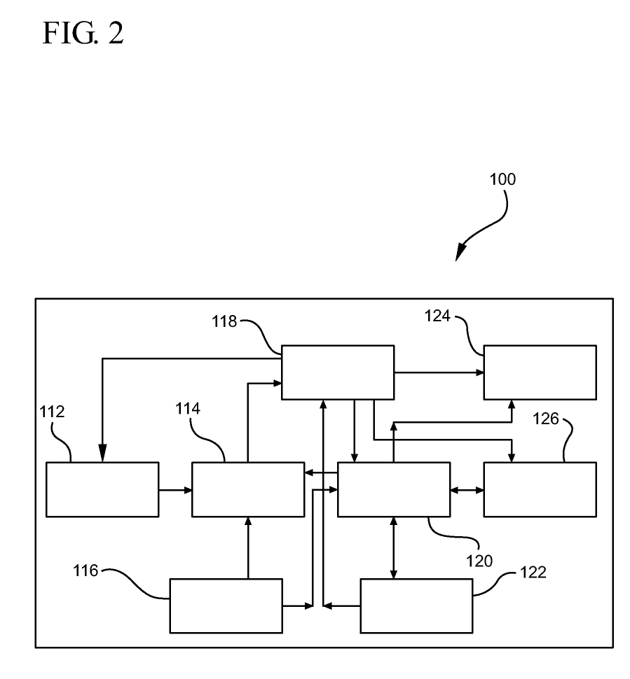

FIG. 1 is a simplified top view depiction of a hand-held test meter 100 with a deep power conservation mode according to an embodiment of the present invention. FIG. 2 is a simplified block diagram of various blocks of hand-held test meter 100.

Once one skilled in the art is apprised of the present disclosure, he or she will recognize that an example of a hand-held test meter that can be readily modified as a hand-hand test meter according to the present invention is the commercially available OneTouch® Ultra® 2 glucose meter from LifeScan Inc. (Milpitas, Calif.). Additional examples of hand-held test meters that can also be modified are found in U.S. Patent Application Publications Nos. 2007/0084734 (published on Apr. 19, 2007) and 2007/0087397 (published on Apr. 19, 2007) and in International Publication Number WO2010/049669 (published on May 6, 2010), each of which is hereby incorporated herein in full by reference.

Hand-held test meter 100 includes a display 102, a plurality of user interface buttons 104, a strip port connector 106, a USB interface 108, and a housing 110 (see FIG. 1). Referring to FIG. 2 in particular, hand-held test meter 100 also includes a battery 112, a first-time-on (FTO) electrical circuit block 114, a buttons electrical circuit block 116, a power supply circuitry block 118, a microcontroller block 120, a communications port block 122, a display control block 124, a memory block 126 and other electronic components (not shown) for applying a test voltage to analytical test strip (not shown), and also for measuring an electrochemical response (e.g., plurality of test current values) and determining an analyte based on the electrochemical response. To simplify the current descriptions, the figures do not depict all such electronic circuitry.

Display 102 can be, for example, a liquid crystal display or a bi-stable display configured to show a screen image. An example of a screen image may include a glucose concentration, a date and time, an error message, and a user interface for instructing an end user how to perform a test.

Strip port connector 106 is configured to operatively interface with the analytical test strip (not depicted in the figures) such as an electrochemical-based analytical test strip configured for the determination of glucose in a whole blood sample. Therefore, the analytical test strip is configured for operative insertion into strip port connector 106. The analytical test strip can be any suitable analytical test strip including an electrochemical-based analytical test strip such as the commercially available OneTouch® Ultra® glucose test strip from LifeScan Inc. (Milpitas, Calif.). Examples of analytical test strips can be found in U.S. Pat. Nos. 5,708,247; 5,951,836; 6,241,862; 6,284,125; 6,413,410; 6,733,655; 7,112,265; 7,241,265; and 7,250,105, each of which is hereby incorporate herein in full by reference.

USB Interface 108 can be any suitable interface known to one skilled in the art. Moreover, USB interface 108 can configured such that battery 112 of hand-held test meter 100 is recharged via USB interface 108 using, for example, recharging techniques that are well known to those of skill in the art. USB Interface 108 is essentially a passive component that is configured to power and provide a data line to communications port block 122 of hand-held test meter 100.

Once an analytical test strip is interfaced with hand-held test meter 100, or prior thereto, a bodily fluid sample (e.g., a whole blood sample) is dosed into a sample-receiving chamber of the analytical test strip. The analytical test strip can include enzymatic reagents that selectively and quantitatively transform an analyte into another predetermined chemical form. For example, the analytical test strip can include an enzymatic reagent with ferricyanide and glucose oxidase so that glucose can be physically transformed into an oxidized form.

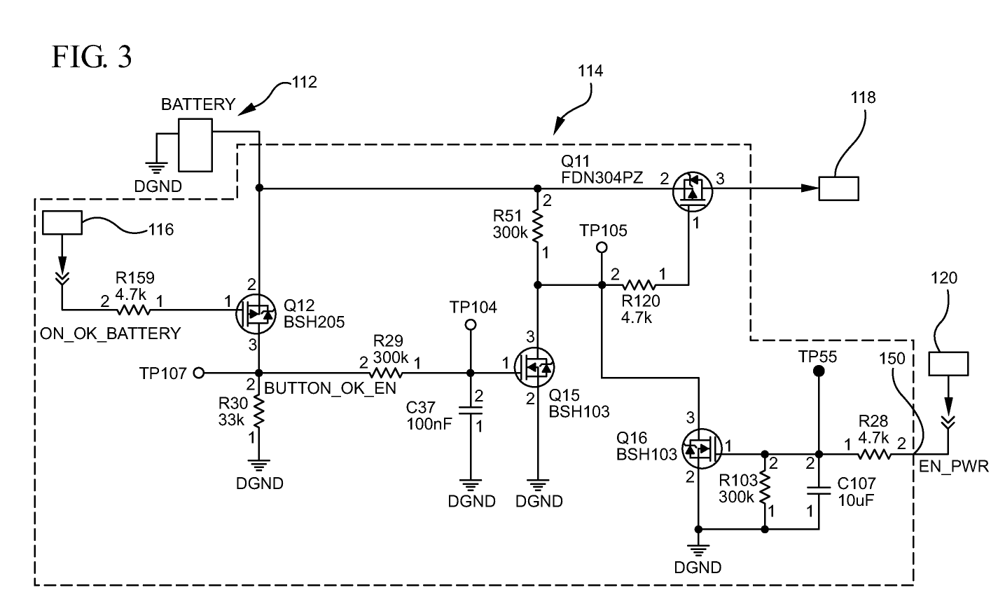

Battery 112 can be any suitable battery including, for example, a rechargeable battery permanently sealed within housing 110. Power supply circuitry block 118 includes, for example, Low Drop-out Regulator (LDO) and voltage regulation circuits well known to those skilled in the art. FTO electrical circuit block 114 is described in detail below with respect to FIG. 3. Memory block 126 of hand-held test meter 100 includes a suitable algorithm that determines an analyte based on the electrochemical response of analytical test strip.

FIG. 3 is a simplified combined electrical schematic and block diagram that depicts a first-time-on (FTO) electrical circuit block 114 in conjunction with battery 112, buttons electrical circuit block 116, power supply circuitry block 118 and microcontroller block 120 as can be employed in embodiments of the present invention.

FTO electrical circuit block 114 is configured to place hand-held test meter 100 into a deep power conservation mode (also referred to as a deep sleep mode) only upon either of (i) the direct application of an electrical signal to the activation node (labeled TP95 in FIG. 3) by an external device or (ii) the receipt of a generated deactivation signal at the signal reception contact 150 (see FIG. 3).

The external device from which the electrical signal is directly applied to the activation node can be, for example, a manufacturing tester that is also employed to test the hand-held meter's functionality during manufacturing and prior to shipment to storage. The deactivation signal (labeled “EN_PWR” in FIG. 3) is generated by microcontroller block 120 in response to an external command signal received by microcontroller block 120 via, for example, USB interface 108.

FTO electrical circuit block 114 is also configured to terminate the deep power conservation mode and place hand-held test meter 100 into a normal operating mode upon receiving a predetermined user triggered signal (labeled “ON_OK_BATTERY” in FIG. 3) from at least one user operable button. The predetermined signal can be generated by any suitable buttons electrical circuit block 116 by, for example, by an end user pushing the OK button depicted in FIG. 1 for at least two seconds. A suitable buttons electrical circuit block is described in co-pending U.S. Patent Application No. 61/359,236. However, once apprised of the present disclosure, one skilled in the art will recognize the deep power conservation mode of hand-held test meters according to embodiments of the present invention can, if desired, also be terminated and the hand-held test meter placed into a normal operating mode via other suitable techniques and configurations. Such techniques and configurations include, for example, those based on the insertion of an external device into USB interface 108.

In the deep power conservation mode, hand-held test meter 100 consumes less than approximately 15 nA of power as power is only being consumed by battery 112 itself through any naturally occurring battery discharge mechanism and momentarily by the buttons electrical circuit block upon pressing of a button and not be any other blocks of the hand-held test meter (such as the FTO electrical circuit block, power supply block, microcontroller block, display control block, communications port block and memory block).

Referring to FIG. 3, the operation of FTO electrical circuit block 114 will now be described in more detail. One skilled in the art the FTO electrical circuit block of FIG. 3 is for descriptive purposes only and that a FTO electrical circuit block employed in embodiments of the present invention can take a form that differs in detail from that of FIG. 3.

When hand-held test meter 100 is in the deep power conservation mode, P-FET transistor Q12 and N-FET transistor Q16 are both inactive and thus the gate of P-FET transistor Q11 is held high (via resistor R91) in a state referred to as an “off” or “disconnect” state. Since transistor P-FET Q11 is in an “off” state, battery 112 is not connected to power supply circuitry block 118 via the path/signal labeled VBAT in FIG. 3 and hand-held test meter 100 is in the deep power conservation mode.

The activation node that places hand-held test meter 100 into the deep power conservation mode when an electrical activation signal is applied directly thereto is labeled TP95 in FIG. 3. Such an applied electrical activation signal can be, for example, a low-level ground (GND) signal or other suitable signal that pulls the gate of N-FET transistor Q16 low, thus deactivating P-FET transistor Q11, and placing hand-held test meter 100 into the deep power conservation mode. In such a deep power conservation mode, microcontroller block 120 is unpowered and thus not capable of generating a high signal at signal reception contact 150 that could inadvertently pull the gate of N-FET transistor Q16 high and disrupt the deep power conservation mode. In the deep power conservation mode, resistor R103 also serves to avoid any inadvertent leakage that could activate N-FET transistor Q16.

As previously noted, the deep power conservation mode can also be entered by the receipt of a deactivation signal at the signal reception contact. In the embodiment of FIGS. 2 and 3, the delivery of a suitable ATE command to microcontroller block 120 (for example, an ATE software command delivered via communications port block 122 to microcontroller block 120) controls the generation of deactivation signal EN_PWR (i.e., the pulling of EN_PWR to a low-level) by the microcontroller block. Such a low-level signal shuts-off (deactivates) N-FET transistor Q16, thus deactivating P-FET transistor Q11, and placing hand-held test meter 100 into a deep power conservation mode. The ATE signal can be any suitable ATE signal known to one skilled in the art designed to control (e.g., initiate) the generation of a deactivation signal by the microcontroller block. It is also noted that the microcontroller block can take any suitable form and include any suitable microcontroller circuitry such as, for example, a microcontroller commercially available from Texas Instruments (Dallas, Tex., USA) as part number MSP430F2618.

The deep power conservation mode is exited upon the application of a predetermined user triggered signal from at least one user operable button (i.e., signal ON_OK_BATTERY in FIG. 3) to the gate of P-FET transistor Q12. P-FET transistor Q12 will thus be pulled low, connecting voltage from battery 112 to the gate of N-FET transistor Q15, via resistor R29. Such a connection of battery 112 to N-FET transistor Q15 activates N-FET transistor Q15 and pulls the gate of P-FET transistor Q11 low, thus providing power from battery 112 to power supply circuitry block 118 and the remainder of the hand-held test meter's blocks, including microcontroller block 120.

Once microcontroller block 120 is powered, microcontroller block 120 is configured to initialize signal EN-PWR of FIG. 3 to a high level. This high level will activate N-FET transistor Q16 which will then also pull the gate of P-FET transistor Q11 low. Hand-held test meter 100 will then remain powered (i.e., not in the deep power conservation mode) as the at least one user activated button is released, as the FTO electrical circuit block remains active due to the presence of a high level signal EN-PWR.

In the FTO electrical circuit embodiment of FIG. 3, both the combination of capacitor C107 with resistor R26, and the combination of resistor R29 with capacitor C37 are configurations that serve as low pass filters that prevent inadvertent changes to the state of the FTO electrical circuit block from, for example, short signals-spikes or ESD-spikes.

In the deep power conservation mode, no power is consumed by the FTO electrical circuit block 114 or other circuit blocks of hand-held test meter 100 other than buttons electrical circuit block 116 in the event a button is pushed. Buttons electrical circuit block 116 is configured to only consume power when a button is pressed, typically for a time period (duration) in the range of milliseconds to a few seconds (i.e., momentarily) to generate the predetermined user generated signal. Buttons electrical circuit block 116, therefore, only consumes an insignificant amount of power. The only notable power consumption in the deep power conservation mode is that associated with natural self-discharge of battery 112, and any battery protection circuit (not depicted in the FIGs.) included in battery 112. During use, FTO electrical circuit block 114 is only powered in its entirety for the few seconds required to terminate the deep power conservation mode by electrically connecting battery 112 to power supply circuitry block 118. However, after the deep power conservation mode has been terminated and microcontroller block 120 has asserted a high level EN-PWR signal, there will be a minimal constant power drain of, for example, 20 μA or less through at least resistor R103.

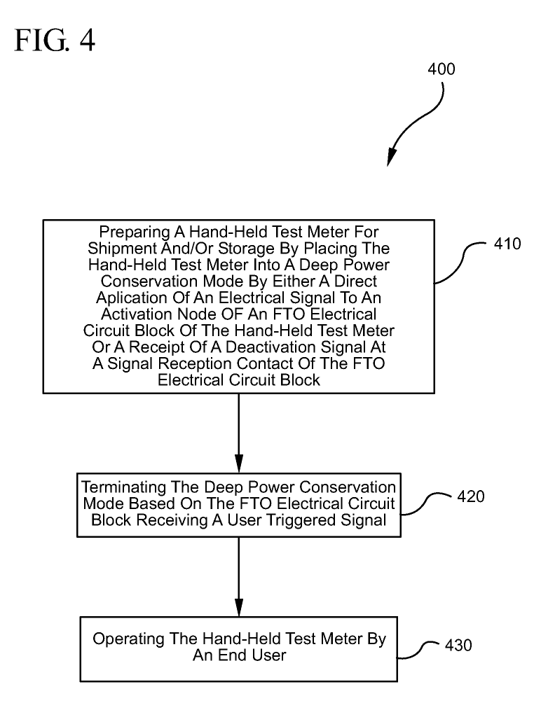

FIG. 4 is a flow diagram depicting stages in a method 400 for operating a hand-held test meter configured for the determination of an analyte (such as glucose) in a bodily fluid sample (e.g., a whole blood sample) according to an embodiment of the present invention. Method 400 includes preparing the hand-held test meter for at least one of storage and shipment prior to end user operation of the hand-held test meter (see step 410 of FIG. 4). The preparation is accomplished by placing the hand-held test meter into a deep power conservation mode via either (i) the direct application of an electrical signal to an activation node of a first time on (FTO) electrical circuit block of the hand-held test meter by an external device (for example, a manufacturing tester employed in a manufacturing process for the hand-held test meter) or (ii) the receipt of a deactivation signal at a signal reception contact of the FTO electrical circuit block.

Method 400 also includes, at step 420, terminating the deep power conservation mode and placing the hand-held test meter into a normal operating mode based on the FTO electrical circuit block receiving a predetermined user triggered signal from a user operable button of the hand-held test meter, and subsequently at step 430 operating of the hand-held test meter by an end user.

In methods according to embodiments of the present invention, the hand-held test meter can be, for example, shipped from a hand-held test meter manufacturing site following the preparing step and prior to the terminating step. In addition, the hand-held test meter can, if desired, be stored following the preparing step and prior to the terminating step. Since the preparing step has placed the hand-held test meter into a deep power conservation mode, such shipping and storage can occur over relatively long durations without complete discharge of a battery included in the hand-held test meter.

Methods according to embodiments of the present invention can, if desired, also include the steps of (i) applying a bodily fluid sample to an electrochemical-based analytical test strip; (ii) measuring an electrochemical response of the electrochemical-based analytical test strip using the hand-held test meter; and (iii) determining the analyte based on the measured electrochemical response.

Once apprised of the present disclosure, one skilled in the art will recognize that method 400 can be readily modified to incorporate any of the techniques, benefits and characteristics of hand-held test meters according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A hand-held test meter for use with an analytical test strip in the determination of an analyte in a bodily fluid sample, the hand-held test meter comprising:
a housing;
a buttons electrical circuit block;
at least one user operable button in operable communication with the buttons electrical circuit block;
a microcontroller block; and
a first-time-on (FTO) electrical circuit block disposed within the housing, the FTO electrical circuit block including an activation node and a signal reception contact, and
wherein the FTO electrical circuit block is configured to place the hand-held test meter into a deep power conservation mode upon either of the direct application of an electrical signal to the activation node by an external device or a deactivation signal received at the signal reception contact, and
wherein the microcontroller block is configured to generate the deactivation signal received at the signal reception contact in response to an external command signal received by the microcontroller block; and
wherein the FTO electrical circuit block is also configured to terminate the deep power conservation mode and place the hand-held test meter into a normal operating mode upon receiving a predetermined user triggered signal from the at least one user operable button.

2. The hand-held test meter of claim 1 further including a communications port block with at least one communication port block input and wherein the signal reception contact is a communication port block input.

3. The hand-held test meter of claim 2 wherein the communications port block is configured to receive the external command and transfer it to the microcontroller block.

4. The hand-held test meter of claim 2 wherein the communications port block is a USB block.

5. The hand-held test meter of claim 1 wherein the FTO electrical circuit block includes at least one low pass filter configured to prevent inadvertent state changes within the FTO electrical circuit.

6. The hand-held test meter of claim 1 wherein the FTO electrical circuit block is configured for low leakage operation by operative integration of at least one resistor.

7. The hand-held test meter of claim 1 wherein the external command signal is an ATE generated software command signal.

8. The hand held test meter of claim 1 wherein the deactivation signal is a low level ground signal.

9. The hand-held test meter of claim 1 further including:
a rechargeable battery disposed within the housing.

10. The hand-held test meter of claim 9 wherein the rechargeable battery is permanently sealed within the housing.

11. The hand-held test meter of claim 1 wherein the hand-held test meter is configured to consume less than approximately 15 nA of power in the deep power conservation mode.

12. The hand-held test meter of claim 1 wherein the hand-held test meter is configured for the determination of glucose in a whole blood sample.

13. A method for employing a hand-held test meter configured for the determination of an analyte in a bodily fluid sample, the method comprising:
preparing a hand-held test meter for at least one of storage and shipment prior to end user operation of the hand-held test meter by placing the hand-held test meter into a deep power conservation mode via either a direct application of an electrical signal to an activation node of a first time on (FTO) electrical circuit block of the hand-held test meter by an external device or a receipt of a deactivation signal at a signal reception contact of the FTO electrical circuit block;
terminating the deep power conservation mode and placing the hand-held test meter into a normal operating mode based on the FTO electrical circuit block receiving a predetermined user triggered signal from a user operable button of the hand-held test meter; and
operating the hand-held test meter by an end user.

14. The method of claim 13 wherein the preparing step includes preparing a hand-held test meter that includes a microcontroller block is configured to generate the deactivation signal received at the signal reception input contact in response to an external command signal received by the microcontroller block.

15. The method of claim 14 wherein the preparing step includes placing the hand-held test meter into a deep power conservation mode via receipt of a deactivation signal generated by the microcontroller block at the signal reception contact of the FTO electrical circuit block.

16. The method of claim 14 wherein the external command signal is an ATE generated software command signal.

17. The method of claim 14 wherein the deactivation signal is a low level ground signal.

18. The method of claim 13 wherein the signal reception contact is a communication port block input of the hand-held test meter.

19. The method of claim 13 further including the step of shipping the hand-held test meter from a hand-held test meter manufacturing site following the preparing step and prior to the terminating step.

20. The method of claim 13 further including the step of storing the hand-held test meter following the preparing step and prior to the terminating step.

21. The method of claim 13 wherein the terminating step occurs based on the FTO electrical circuit block receiving a predetermined user triggered signal from a user operable button of the hand-held test meter.

22. The method of claim 13 further including:
applying a bodily fluid sample to an electrochemical-based analytical test strip;
measuring an electrochemical response of the electrochemical-based analytical test strip using the hand-held test meter; and
determining the analyte based on the measured electrochemical response.

23. The method of claim 13 wherein the bodily fluid sample is a whole blood sample and the analyte is glucose.
